(19) United States
(12) Patent Application Publication
(10) Pub. No.: US 2009/0053818 A1
(43) Pub. Date: Feb. 26, 2009

(54) Quantitative proteomics with isotopic substituted raman active labeling

(76) Inventors: Dongmao Zhang, Houston, TX (US); Jo V. Davisson, West Lafayette, IN (US); Dor Ben-Amotz, West Lafayette, IN (US); Yong Xie, Thousand Oaks, CA (US); Kumar Shirshendu Deb, West Lafayette, IN (US)

Correspondence Address: COLEMAN SUDOL SAPONE, P.C., 714 COLORADO AVENUE, BRIDGE PORT, CT, 06605-1601, US

(21) Appl. No.: 11/663,862

(22) PCT Filed: Sep. 26, 2005
(86) PCT No.: PCT/US05/34795

§ 371 Date: Oct. 15, 2007

Related U.S. Application Data
(60) Provisional application No. 60/613,378, filed on Sep. 27, 2004.
(60) Provisional application No. 60/647,046, filed on Jan. 26, 2005.
(60) Provisional application No. 60/661,202, filed on Mar. 11, 2005.

(51) Int. Cl. G01N 33/68; C07D 311/82; G01N 33/50

(52) U.S. Cl. 436/86; 549/394; 436/94

(57) ABSTRACT

A labeling reagent having a distinct Raman, or surface enhanced Raman, spectral signature is used for the control and analysis samples. The labeling reagents can be fluorescent dyes with different isotopic substituents, such as the substitution of some hydrogen atoms for deuterium atoms. Such labeling does not have any detectable effect on separation retention. Raman spectroscopy is used for detection purposes. By combining SERS and SERRS, a concentration ratio prediction error of less than 3% can be obtained over four orders of magnitude of total concentration with up to a factor of 3 concentration ratio range. The method is reliable, reproducible and more sensitive than methods based on absolute SERS/SERRS intensity correlations, with no internal standard, or using a different molecule (rather than an IEIS) as a SERS/SERRS internal standard.

### STATEMENT REGARDING FEDERALLY SPONSORED RESEARCH OR DEVELOPMENT

The U.S. Government has a paid-up license in at least parts of this invention and the right in limited circumstances to require the patent owner to license others on reasonable terms as provided for by the terms of Grant No. GM 153155 of the National Institute of Health.

### BACKGROUND OF THE INVENTION

This invention pertains to the advantageous combined use of isotopic substituted labeling reagents (ISLR), with surface enhanced Raman (SERS) or surface enhanced resonance Raman spectroscopic (SERRS) techniques, and various separation methods for quantitative proteomic studies. The separation methods can include high performance liquid chromatograph (HPLC), gel electrophoresis (2D-PAGE), antibody arrays or aptamer arrays, DNA micro array techniques for determination of gene expression patterns, and other separation methods.

Previous proteomics quantitative methods are generally based on the combination of the isotopic labeling of the control and analysis samples, HPLC separation and mass spectrometer detection. Previous methods generally required labels with a significant mass difference; namely, sufficient difference for independent mass spectral detection of the relative concentrations of two isotopic species. (See, for example, Washburn, M, P., et al. “Analysis of Quantitative Proteomics Data Generated via Multidimensional Protein Identification Technology”. Anal. Chem. 2002, 74, 1650-1657; and Ji. J., et al. J. Chromatogr. B2000, 745, 197-210.) Such mass differences can also produce the unwanted consequence of altering the relative separation retention time of the control and analysis analytes during the HPLC or other separation. Previous methods also typically used mass spectrometry to identify and quantify protein pairs from control and analysis samples.

With previous methods, the protein quantification for the control and experimental sample are often done at the peptide levels because of the limitation of the detection method, tagging method and the separation method. With those methods, the mass difference introduced with the isotopic labeling have to be large enough to enable the differentiation of the peak clusters of the peptide pairs from the control and analysis sample. It is demonstrated in the literature that there is considerable retention time difference once that mass difference is larger than about 4 mass units. (See, for example, Zhang, R., et al. “Fractionation of Isotopic Labeled Peptides in Quantitative Proteomics” Anal. Chem. 2001, 73, 5142-5149.) With previous methods, each peptide pair has different characteristics and has multiple set of peaks. Thus, data analysis has to be done peptide by peptide. Although theoretically, multiple samples can be analyzed simultaneous by introducing larger and larger mass shift, the limitations of such simultaneous analysis are apparent.

Previous comparative gene expression methods are based on (a) the combination of fluorophore labeling and fluorescence detection (Yang Y. H., et al. Normalization for cDNA Microarray Data: A Robust Composite Method Assessing Single and Multiple Slide Systematic Variation. Nucl. Acid Res. 2002, 30:e15), and (b) radioactive labeling and imaging (Salin H., et al. A Novel Sentive Microarray Approach for Differential Screening Using Probes Labeled with Two Different Radioelements. Nucl. Acid Res. 2002, 30:e17). In practical applications, the fluorescence labeling method is the most commonly employed. Previous methods generally required sufficient structural differences in the labeling fluorophores so that the fluorescence signal of the fluorophore can be excited and/or detected at different wavelengths. However, the structural differences can produce different incorporation rates in the direct and indirect labeling methods (Yu J., et al. Evaluation and Optimization of Procedures for Target Labeling and Hybridization of cDNA Microssarys., Mol. Vis. 2002, 8:130-137). The different incorporation rates male the data analysis more difficult and more problematic. It would be desirable if the structural differences and different incorporation rates could be eliminated, or at least minimized.

Conventional fluorescence labeling or tagging methods detect and quantify genes from different samples through fluorescence intensity excited and/or detected at different wavelengths. In general, the methods require multiple excitation lasers and detection filters. Additionally, factors such as bias in the efficiency of dye incorporation, fluorescence background at substrates, imperfection of the optics and the detector, fluorescence quenching, and different quantum efficiency of the labeling dyes at different densities will deteriorate the quantification accuracy. To alleviate this problem, it is generally necessary to derive a normalization procedure, which assumes that most of the genes from different samples will have the same expression pattern. However, even with this normalization, the detection of gene expression of less than 2 folds remains a great challenge. However, it is reported, and generally accepted, that even small modulation of gene expression can be of major biological significance. It would be desirable if the duplicate equipment requirements could be avoided. It would be additionally desirable if normalization procedures that may mask small modulations of gene expression could be avoided.

There is a significant amount of previous work done on Raman labeling and SERRS or SERS detection for protein, antibody, and DNA. (See, for example: (a) Grubisha, D. S., et al., “Femtomolar Detection of Prostate-specific antigen: An Immunoassay Based on Surface-Enhanced Raman Scattering and Immunogold labels”. Anal. Chem. ASAS publication. (b). Mirkin C. A. et al. US Patent application 20030211488. (c) Cao, Y. C., et al. “Nanoparticles with Raman Spectroscopic Fingerprints for DNA and RNA Detections” Science, 2002, 1536-1540. (d). Graham, D., et al. “Surface-Enhanced Resonance Raman Scattering as a Novel Method of DNA Discrimination”. Angew. Chem. Int Ed. 2000, 39(6), 1061-1063.)

Also, it is known that the total concentration of labeled dye molecules can be easily determined using standard UV-VIS absorption or fluorescence methods, or with a SERS or SERRS signal. With the latter, the dynamic range is determined to be about 4 orders of magnitude from 10⁻¹¹M to 10⁻⁷M under optimal conditions. (See, for example, D. Graham, et al., Anal Chem., 1997, 69, 4703.)

Since the discovery of SERS (Fleischmann, M., et al., Chem. Phys. Lett. 1974, 26, 163) various SERS active substrates and molecules have been reported with a typical Raman signal enhancement of 10⁶. Under SERRS conditions far greater enhancements may be attained, and single molecule detection limits have been reported for Rhodamine 6G, adenine, cresyl violet and other SERRS active molecules (Kneipp, K., et al., Phys. Rev. E 1998, 57, R6281-4; Koo, T.-W., et al., Appl. Spectrosc. 2004, 58, 1401-7; Nie, S., et al., Science 1997, 275, 1102-6; Kneipp, K., et al., Phys. Rev. Lett. 1997, 78, 1667-70). Because of the high sensitivity of the SERS/SERRS techniques and high information content of the resulting vibrational spectra, SERS active molecules have been employed as labeling reagents for bioanalytical applications which enabled detection of a mol (10⁻¹⁸ mol) quantities of proteins or DNAs down to fM (10⁻¹⁵ mol/l) concentrations (Cao, Y. C., et al., Science 2002, 297, 1536-40; Faulds, K., et al., Analyst 2004, 129, 567-8; Graham, D., et al., Anal. Chem. 2002, 125, 1069-74; Cao, Y. C., et al., J. Am. Chem. Soc. 2003, 125, 14676-7). However, accurate quantitative analysis with SERS remains a challenge because of (1) the difficulties associated with the production of reproducible SERS active substrates, (2) the strong dependence of the SERS enhancement on the distance between the analyte and the SERS substrates (Lacy, W. B., et al., Anal. Chem. 1999, 71, 2564-70), (3) variations of SERS enhancement with on the surface coverage of the analyte on the substrate (related to the distribution of SERS active hot-spots) (Campion, A., et al., Chem. Soc. Rev. 1998, 27, 241-50). In addition, quantitative concentration measurements using optical methods (including SERS as well as normal Raman or fluorescence) must contend with intensity variations produced by changes in excitation and/or collection efficiency. Correcting for such variations is most often accomplished using either an internal or an external standard to calibrate the correlation between the optical signal and the concentration (or amount) for the analyte of interest.

For example, as reported in Anal. Chem. 2002, 74, 3160-7, Smith et al. employed a flow-cell device for in-situ aggregation of Ag colloidal fabricated with the Lee-Misel method (J. Phys. Chem. 1982, 86, 3391-95), and found that good linearity and reproducibility when using the same batch of a SERS active colloidal solution. However, when different batches of colloidal solutions were used, the reproducibility of the SERRS signal with mitoxantrone concentration deteriorated significantly with calibration slope differences of up to 60%, even though all the other experiment conditions remained the same (McLaughlin, C., et al., Anal. Chem. 2002, 74, 3160-7). More recently, an internal standard method was proposed to improve the accuracy for SERS (colloidal) quantification by using the SERS signal generated from a self-assembled monolayer (SAM) as an internal standard (Loren, A., et al., Anal. Chem. 2004, 76, 7391-5). With this method, the high coverage of the SAM is presumed to prevent chemisorption of the analyte onto the SERS active surfaces and thus to improve reproducibility.

However, this SAM approach also has intrinsic limitations. For example, because of the sharp drop-off of the SERS enhancement with the distance between the analyte and SERS surface, the limit of the detection and the dynamic range with the SAM approach has been severely compromised (because of the greater distance between the analyte and SERS surface created by the SAM coating). Furthermore, the different local environments around the SAM and the analyte molecules may produce a different response to experiment parameters such as laser intensity and frequency. These and other factors may explain the relatively large prediction errors (Root mean prediction error of 0.5 μM for samples between 0.1 μM and 5 μM) observed by Loren, et al., when using this SAM internal standard method. What is needed is a reliable method which may be used for quantitative SERS/SERRS measurements over a wide concentration range with unprecedented accuracy and reproducibility.

Biomarkers are molecules (such as particular protein or DNA structures) which are correlated with the onset of a particular disease/health state. Previous detection and quantification methods for biomarker detection include (a) fluorescence tagging based approach, (b) surface plasmonic resonance analysis and localized surface plasmonic resonance analysis (Haes, A. J., et al., “Detection of a Biomarker for Alzheimer's Disease from Synthetic and Clinical Samples Using a Nanoscale Optical Biosensor” J. Am. Soc. 2005 ASAP-publication). However, fluorescence methods suffer from a relatively small dynamic range (four orders of magnitude, or smaller, in concentration) and large quantification error (caused by photo-bleaching and imperfection of the assay substrates). Surface plasmon resonance based methods require lengthy incubation time allowing antibody to capture the biomarkers, which could cause biomarkers degradation, furthermore, these methods requires intensive calibration. What is needed is a reliable method for detecting biomarkers of interest over a wide concentration range.

### SUMMARY OF THE INVENTION

With this invention, a labeling reagent is used that has a distinct SERS or SERRS spectral signature. Such labeling can be done in such a way as not to have any detectable differential effect on separation retention or the binding affinities of the analytes of interest. The labeling reagents used for this invention can be, for example, dyes with different isotopic substituents, such as the substitution of some hydrogen atoms for deuterium atoms. Other isotopic substitutions may achieve sufficiently distinctive SERS or SERRS spectra. The substitution can be employed in such SERS or SERRS active dyes such as xanthene dyes like Rhodamine and Fluorescein, triarylmethane dyes like Cresyl Violet, azo dyes like Benzotriazole azo, mercaptopyridine, and others. The isotopic variants of these and other dyes can be obtained through the use of isotopically substituted precursors that are then used during the dye-forming condensation reaction. The isotopic variants may also be obtained by isotopic exchange of the labile aromatic protons of the chromophore by heating the dye in a deuterated acidic media.

In one aspect of this invention, proteins, peptides, cDNAs or other analytes from control and analysis samples can be labeled, for example by covalent attachment, directly or indirectly, or Genisphere labeling and TSA methods, with SERS or SERRS active dyes which only differ by isotopic substitution. Following the addition of the isotopically substituted SERS or SERRS active reagent to samples containing analytes of interest, the mixture of two or multiple samples can be subjected to (1) 2D-PAGE, HPLC or other separation techniques, or (2) the antibody array or aptamer arrays, and their SERS and/or SERRS spectra can be detected with a Raman spectrometer, Raman microscope or Raman imaging system. Comparable characteristics of the control and analyte samples can be deduced from the SERS or SERRS signatures using known data analysis algorithms.

Some differences between the present invention and previous methods are (1) the type isotopic labels used, (2) the detection method used, (3) the special characteristics of the labeling dyes used, (4) the improved separation retention characteristics obtained, (5) the more efficient data analysis scheme enabled, (6) and the multiplexing capability permitting multiple samples to be analyzed. In one example, the protein pairs refer to the same protein from the control and analysis samples, and the analysis samples can be multiple as demonstrated in point (6). In addition to the capability of top-down analysis, the current approach enables bottom-up proteomics approach in which proteins are analyzed without digestion. In another example, the labeling reagents for genes from different samples differ only in isotopic substitutions. Thus the incorporation bias is minimized, which enables more accurate comparative quantification of genes from different samples.

These dye molecules may advantageously have an affinity to a SERS active surface and thus have extremely high SERS or SERRS cross-section. In fact, the Raman signal of the labeling reagents can be so strong that it dwarfs the spectral contribution from the proteins or peptides to which the labeling reagent are bound. This fact can be advantageously be utilized for efficient data collection and analysis since the signal of interest can be collected more rapidly with little or no interfering background signals. Furthermore, the Raman spectra can be obtained with directly coupled separation instruments. The relative quantities of the protein or peptide pairs can be obtained by comparing the Raman signal from isotopic substitute labeling reagents present in the same chromatographic separation fraction.

The chemical characteristics of the labeling reagents used for the present invention are generally the same, and so are their SERS or SERRS detection schemes and devices. The labeling reagents used for present invention can be dyes with strong absorbance at visible wavelengths and high quantum yield of fluorescence. The total concentration of the labeled dye molecules can be easily determined using standard UV-VIS absorption or fluorescence methods or with a SERS or SERRS signal. Thus, the relative quantification of the signals from different tags will be immune from most of the adverse factors mentioned with respect to other label detection methods. Furthermore, since the complete SERS or SERRS spectra can be subjected to data analysis, the interference from background noise can be greatly reduced with advanced multivariate data analysis algorithms such as partial least square methods or neural networking methods. Thus, as demonstrated with the preliminary data, the quantification accuracy with this present invention is much higher than those obtained with previously employed methods.

For example, the SERS or SERRS signal derived from different labels can enable a determination of the relative ratio of proteins from control and experimental samples. Thus, with the current invention, the absolute quantity of proteins can be obtained once the stoichiometric relationship is known for the labeling reactions. Furthermore, with the current invention, the protein quantification can be done at the protein level, thus the relative mass difference is much smaller when the same absolute mass difference is produced with isotopic labeling, which in turn, will guarantee the retention time difference be negligible for the protein pairs from the control and experimental sample.

In accordance with one aspect of the current invention, the detected signal from the separated protein pairs are from the isotopic substituted labeling reagent (ISLR) pairs, not from the proteins being labeled. Thus, once the analysis scheme is derived for one pair of the proteins, it is applicable universally for all the proteins samples labeled with same ISLRs. As a further example, the detected signal from the separated cDNA pairs are again from the ISLR pairs, not from the genes being labeled. Thus, once the analysis scheme is derived for one gene expression, it is applicable universally for all the genes labeled with same ISLRs. With present invention, multiple samples can be analyzed by performing isotopic substitution at different positions or on functional groups with the same labeling reagents, and the different ISLRs of the same labeling reagents can be of the same mass or slightly different masses. Thus, with current invention, multiple samples can be analyzed without the concern of difference in the separation retention time and the difficulties of the data analysis. This invention can be much more sensitive than the detection methods used in the prior art since the SERS or SERRS spectra of the dye can be easily obtained with concentration <10 pM with a sample volume of <1 uL in the solution phase. It also has greater dynamic range since that high quality spectra have been obtained with concentration up to 10 uM as shown in the Description of Illustrative Examples.

When the separation is performed with HPLC, the SERS or SERRS acquisition can be coupled with HPLC as a detector, and the colloidal SERS substrate, such as a silver or gold nano-particle suspension, can be introduced either by mixing with the eluted fractions or by mixing with solvent. If the separation is done with 2D-PAGE, the colloidal substrate can be applied by staining of the gel or the membrane with colloidal particles as suggested by Cao Y. C, et al., Raman Dye-Labeled Nanoparticle Probes for Proteins. J. Amer. Chem. Soci. 2003 ASAP publications. If the ISLR labeled protein pairs are separated with the antibody or aptamer arrays, SERS active Ag colloidal will be introduced into the array substrates after washing off the nonspecific bounded proteins.

According to the present invention, an isotopically edited internal standard (IEIS) method, which may be used for quantitative SERS/SERRS measurements over a wide concentration range with unprecedented accuracy and reproducibility, employs standard molecules that have virtually identical chemical properties to the analyte molecule. As a result, their relative SERS/SERRS intensity is far less sensitive to batch-to-batch colloid solution variations, optical excitation/collection parameters. The present method differs in other important ways as well.

In general the SERS signal intensity, I, can be a function of several variables as shown in equation 1:

I_{κ}^{δ}=C_{κ}I_{κ}^{s}αₖ Eq. 1

Where I_{κ}^{δ} is the signal intensity obtained from sample κ of concentration of C_{κ} under a given set of experiment conditions, δ, while I_{κ}^{s} represents the signal intensity obtained with unit analyte concentration under a given set of standard conditions, s, and α_{κ} represents the relative SERS enhancement factor, which may in general also be a function of C_{κ}, as well as the characteristics of the SERS substrate and Raman system.

For an internal standard to be effective, all the SERS intensity variations introduced by anything other than sample concentration should be compensated by internal SERS intensity standard. In other words, an ideal internal standard method is one for which the SERS intensity ratio of the analyte, a, and internal standard reference, r, are strictly proportional to the ratio of their corresponding concentrations. That is:

$$\frac{{I}_{a}^{δ}}{{I}_{r}^{δ}} = \frac{{C}_{a} {I}_{a}^{s}}{{C}_{r} {I}_{r}^{s}} \qquad Eq . 2$$

Ideally, the relative enhancement factors of the analyte and reference compound should be identical, αₐ=αᵣ, and this equality should not depend on either the absolute or relative concentrations of the analyte and reference compounds, or any other experimental variables. This implies that the ideal internal standard should have chemical properties which are as similar as possible to the analyte of interest. However, in order to be able to differentiate and quantify the spectral contribution for the analyte and its internal standards, their SERS spectral features have to be sufficiently different to facilitate independent measurement of their SERS intensities in a mixture.

Given the above consideration, one might expect that an ideal internal standard for any analyte would be an isotopically edited version of the analyte of interest. In fact, isotope editing is a commonly used technique in vibrational spectroscopy, to aid in assignment of spectroscopic features associated with specific functional groups. Advantages of isotopic editing as an internal standard method include the fact that the two compounds are expected to have (a) virtually the same chemical and physical properties but (b) a readily measurable and quantifiable spectroscopic differences. The fact that isotope editing can produce significant spectral changes is illustrated, for example, by the observed peak red-shifts of 50 and 30 cm⁻¹ observed in the vibration of the ring breathing mode of benzene produced H² (D) or C¹³ editing, respectively (Shimanouchi, T., Tables of Molecular Vibrational Frequencies, National Bureau of Standards, 1972; Painter, P. C., et al., Spectrochimica Acta 1977, 33A, 1003-18), which are quite significant given that corresponding Raman band has a full width at half maximum of <6 cm⁻¹. Furthermore, since most molecules with large SERS or SERRS activities contain aromatic functional groups and the Raman signals of these functional groups are in general the most prominent features in the resulting Raman spectra, isotopic editing of aromatic groups should provide a widely applicable IEIS method.

Like other internal standard methods, SERS quantification with IEIS is carried out by mixing the sample of interest with its IEIS of known concentration before incubation of the mixture with a SERS active substrate (e.g. a colloidal solution). After SERS acquisition, the concentration ratio of the analyte and the internal standard may thus be determined from the ratio of the spectral features associated with the two compounds.

The SERS and SERRS spectroscopy of Rhodamine 6G (R6G) has been extensively studied, (See, for example, Bosnick, K. A., et al., J. Phys. Chem. B 2002, 106, 8096-9; Li, G., et al., Chem. Phys. Lett. 2000, 330, 249-54) as it is one of the most commonly used SERS and SERRS tags in DNA and protein detections applications, (Graham, D., et al., Analyst 2003, 128, 692-9) as well as in SERRS single molecule detection studies. Thus, R6G is a model compound to demonstrate the feasibility and performance of IEIS for SERRS and SERS quantitative analysis. In addition, in order to investigate the feasibility using molecules other than R6G for IEIS, we have also investigated the SERS spectra of mixtures containing both R6G and adenine. The latter studies detailed below also serve to clearly demonstrate the superior performance of the IEIS method as opposed to the use of chemically different analyte and internal standard compounds. The robustness and accuracy of SERS and SERRS with IEIS was evaluated using several batches of colloidal solution, with R6G concentrations varying from 200 pM to 2 μM. Potential biomedical applications with IEIS are also contemplated by this invention.

Additional features and advantages of the present invention will become apparent from the following discussion of exemplary embodiments that are merely intended to be illustrative and not limiting on the scope of the present invention.

### BRIEF DESCRIPTION OF THE DRAWINGS

FIG. 1 is a schematic diagram representing the synthetic procedure used to produce for Rhodamine 6G (R6G) with no deuterium (R6G-d0), and with 4 deuterium substitutions (R6G-d4).

FIG. 2 is a graph of the SERRS spectra of (a) R6G-d4 and (b) R6G-d0 at a concentration of 1×10⁻¹⁰M. The SERS spectra of (c)R6G-d4 and (d) R6G-d0 were obtained at concentration of 10 uM. The SERRS spectra were obtained with an integration time of 15 seconds at a power of 33 mW obtained with Argon ion laser (514 nm). The SERS spectra were obtained with 12 mW of HeNe laser (632.8 nm) with integration time of 0.1 s. It should be noted that SERS spectra of R6G-d0 and R6G-d4 can also be readily obtained at 1 nM (data not shown).

FIG. 3 is a SERRS spectra R6G-d0 and R6G-d4 mixtures at different total concentrations specified at the bottoms of each plot, while the ratio of R6G-d4/R6g-d0 are specified at the right margin.

FIG. 3A is a SERS spectra obtained from solutions each of which has 50/50 R6G-d0/R6G-d4 concentration ratio but different total R6G concentrations: (a) 20 nM, (b) 200 nM, (c) 2 μM (the spectra are offset for clarity).

FIG. 4 is a prediction of the ratio of R6G-d0 vs. R6G-4d based on the spectral signature of the mixture with theoretical compositions shown as values in the x-coordinate.

FIG. 5 is a SERRS spectra of R6G-d0. Spectra (a), and (b) are obtained at R6G-d0 concentration of 100 μM with a water solvent (a), and a mixture of acetonitrile/water (25/75) (b), respectively. Spectrum (c) is obtained by depositing 4 ul of the solution for spectra (b) onto a quartz substrate.

FIG. 6 is a SERS spectra taken when R6G-d4 was added into a premixed R6G-d0/Ag colloidal solution with the same R6G-d0 and R6G-d4 concentrations. Spectra (a)-(d) were obtained at 0 min, 1 min, 80 min and 290 min after adding the R6G-d4. Spectrum (e) was acquired from a solution in which R6G-d0 and R6G-d4 were pre-mixed before adding the Ag colloid solution.

FIG. 7 is a SERS spectra obtained with (a) pure R6G-d0, (b) pure adenine, (c) 10 μM adenine and 100 nM R6G-d0, (d) 1 μM adenine and 10 nM R6G-d0, and (e) 100 nM adenine and 1 nM R6G-d0. The 615 cm⁻¹ peak intensities were adjusted to the same value in spectra (c)-(e) for to better visualize the relative intensity differences of the adenine and R6G features although all three spectra have the same adenine/R6G concentration ratio of 100/1.

### DESCRIPTION OF ILLUSTRATIVE EXAMPLES

All the reagents used for organic synthesizing and colloidal solution and adenine were of analytical grade (Sigma-Aldrich). High purity water (Millipore) was used in all working examples. Silver colloidal solution was synthesized according to the Lee-Misel method by citrate reduction of silver nitrate. Five batches of colloidal solution were synthesized independently and the first 3 batches were used for SERRS measurement and the last two for SERS. The aging period for different batches of colloidal solution varied from 2 hours to 4 days.

The SERS spectra were obtained using a home-built micro-Raman system with a 632.8 nm HeNe laser (with 10 mW at the sample), while the SERRS measurements were performed with another home-built Raman system with 514 nm argon ion excitation lasers (with 6 mW at the sample). With both systems, the back-reflected Raman signal was collected using a 20× Olympus objective and coupled to a spectrograph with a fiber-bundle for detection with a liquid-nitrogen cooled CCD detector. The spectrograph used in the 633 nm system is equipped with a He—Ne laser and a 1200 gr/mm grating, while that in the 514 nm system is equipped with an Ar-ion laser and a 1200 gr/mm grating.

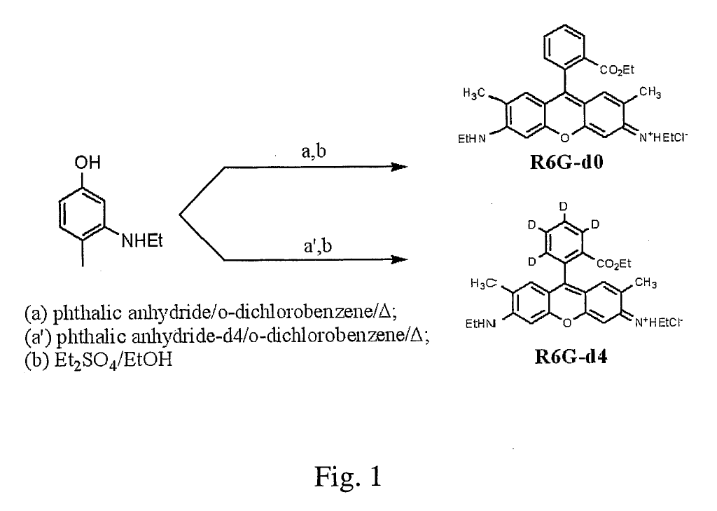

R6G and its IEIS derivative were synthesized by coupling 3-(ethylamino)-4-methyl phenol with commercially available phthalic anhydride and d4-phthalic anhydride respectively, followed by ethylation of the free carboxylic acid groups. The synthetic route is illustrated in FIG. 1. Since four H atoms are substituted with D in the isotopically edited R6G, the two compounds will from hereon be abbreviated as R6G-d0 and R6G-d4, while the term R6G will continue to be used to refer to either one or both of the isotopes.

All the SERRS experiments with R6G were repeated three times, each with a different batch of silver colloidal solution. The samples used for each repeated trial were the same: the total R6G concentration varied from 200 pM to 200 nM, quantitative SERRS analysis with IEIS was only carried out at two concentrations of 200 pM and 200 nM. For the SERS measurements, the final concentration of Rhodamine 6G varied from 20 nM to 2 μM, and the same set of samples were analyzed with two batches of colloidal solutions. For all the IEIS samples, the concentration ratio of R6G-d0 to R6G-d4 was 100/0, 75/25, 66.7/33.3, 50/50, 33.3/66.7, 25/75 or 0/100.

If not otherwise specified, all SERS/SERRS measurements were performed using the following procedure: after mixing of 2 mL silver colloidal solution with 2 mL of high purity water in a 5 ml glass vial, 200 μL of 2% NaCl solution was added for aggregation followed by immediate addition of 300 μL of analyte solution. The prepared sample was allowed to sit for 2 minutes before spectral acquisition. The integration times for all SERRS and SERS measurement are listed in Table 1, no attempt was made to optimize the signal intensities for each measurement.

TABLE 1
Integration times for SERS and SERRS measurements

| | Excitation | | | | | | | | |
|---|---|---|---|---|---|---|---|---|---|
| Silver | laser | | Total Concentration of R6G-d0 and R6G-d4 | | | | | | |
| Colloidal | nm | mW | 200 pM | 2 nM | 20 nM | | 200 nM | | 2 μM |
| Batch one | 514 | 10 | 30 s | 5 s | 500 | ms | 50 | ms | |
| Batch two | 514 | 10 | 30 s | 5 s | 500 | ms | 50 | ms | |
| Batch | 514 | 10 | 10 s | 5 s | 500 | ms | 10 | ms | |
| three | | | | | | | | | |
| Batch four | 633 | 6 | | | 5 | s | 1 | s | 200 ms |
| Batch five | 633 | 6 | | | 5 | s | 1 | s | 100 ms |

For all the spectral analysis, the following simple least square spectral decomposition algorithm was developed and implemented using Matlab (MathWorks Inc.) to calculate the relative concentration ratio of R6G-d0 and R6G-d4. Letting S₁ and S₂ stand for the SERS/SERRS spectra obtained with pure R6G-d0 and R6G-d4 respectively, and D for the SERS/SERRS spectrum measured with mixture of an unknown quantity of R6G-d0 with R6G-d4 of concentration Cᵣ. Since spectrum D contains only spectral contribution from S₁ and S₂ plus experiment noise, spectrum D can be decomposed into spectra S₁ and S₂ by solving the Equation 3 with the least square method:

$$\vert {C}_{1} {C}_{2} \vert \vert \begin{matrix}{S}_{1} \\ {S}_{2}\end{matrix} \vert = D \qquad Eq . 3$$

However, it should be noticed that C₁ and C₂ are not the concentrations of S₁ and S₂, in fact, they don't even represent their relative contributions to mixture spectrum D before a proper adjustment of spectral intensity of S₁ or S₂. The goal for this adjustment is to make the intensity ratio of the component spectra equal to what would be obtained with the component spectra each acquired under exactly the same conditions. This can be done by simply finding a multiplying constant for S₁ or S₂ so that the C₁/C₂ ratio determined with the adjusted component spectra matrix will be equal to 1 for any SERS/SERRS spectra obtained with the mixture consisting of exactly 50% of each component, and the resulting intensity-calibrated component spectra are used for all subsequent spectral analysis.

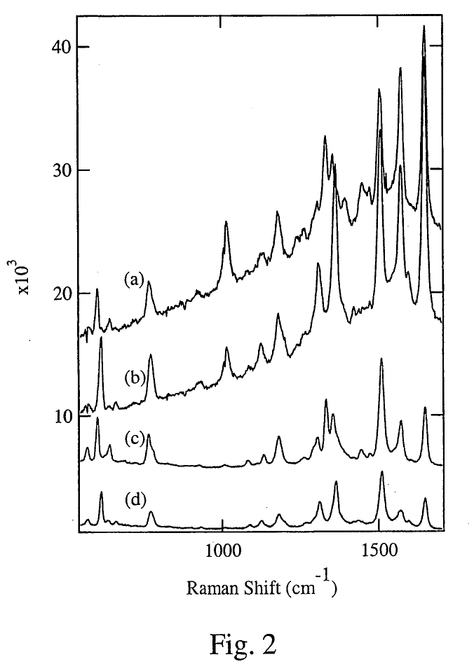

To demonstrate the capability of detection and quantization of ISLR pairs with SERS or SERRS spectra in large concentration range, SERS and SERRS spectra of both reagents are obtained at a concentration of 1×10⁻⁵M, 1×10⁻¹⁰M respectively. The spectra are shown in FIG. 2. The observed spectra are vertically shifted to permit easy comparison. As evident in both the SERRS and SERS spectra, several Raman bands are red-shifted in R6G-d4 relative to their locations in R6G-d0, which is consistent with the higher mass of deuterium relative to hydrogen. The peaks at 615 cm⁻¹ (ring in plane bending) and the 771 cm⁻¹ (C—H bending) of R6G-d0 are shifted to 604 cm⁻¹ and 763 cm⁻¹, respectively, in R6G-d4. Interestingly, the singlet peak at 1356 cm⁻¹ (aromatic C—C stretch) of R6G-d0 splits into to peaks at 1325 cm⁻¹ and 1350 cm⁻¹ in R6G-d4. (Assignments of bending and stretch per: Li, G. et al., Chem. Phys. Lett. 2000, 330, 249-54).

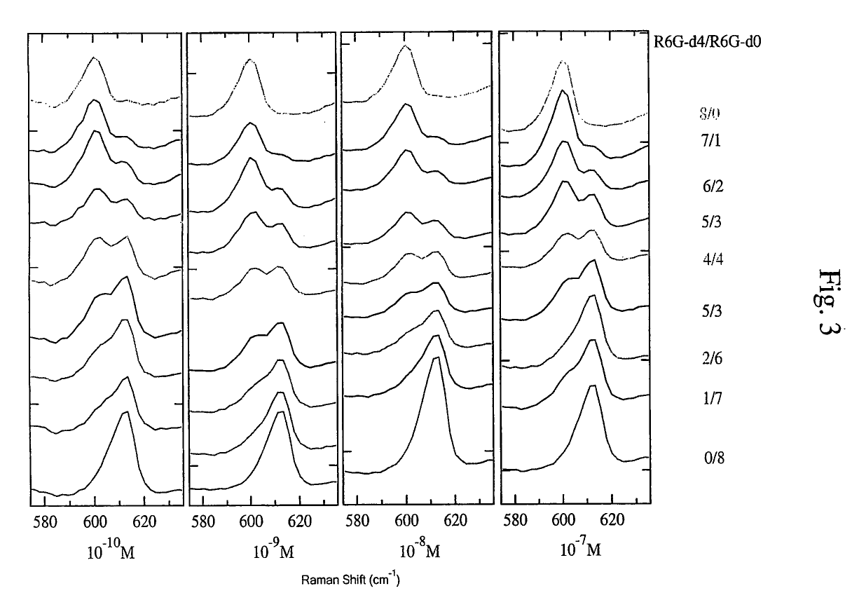

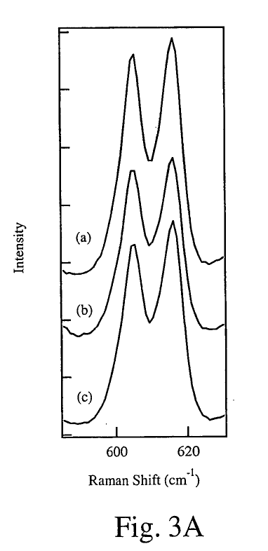

Careful examination of the R6G-d0 and R6G-d4 SERS or SERRS spectra shown in FIG. 2 reveals spectral differences in the regions of 575 cm⁻¹ to 635 cm⁻¹, 1280 cm⁻¹ and 1380 cm⁻¹. To determine whether these differences are significant enough to quantify the relative quantity of R6G-d0 or R6G-d4 in their mixture, SERRS spectra of solution mixtures with different ratios of both samples are taken. The spectral region of 575 cm⁻¹ to 635 cm⁻¹ is shown in FIG. 3. The total concentrations are specified at the bottoms of each plot. Each plot shows nine different relative concentrations that are displayed as the nine different curves. The ratios of R6G-d0/R6g-d4 are 0/8, 1/7, 2/6, 3/5, 4/4, 5/3, 6/2, 7/1, 8/0 from top curve to bottom curves.

To demonstrate IEIS can also be used for SERS quantification, similar experiment procedures were applied for SERS spectral acquisition and analysis. FIG. 3A shows the SERS spectra obtained from samples with same R6G-d4/R6G-d0 ratio of 50/50, but different total R6G were (a) 20 nM, (b) 200 nM, and (c) 2 μM. It can be seen that the relative spectroscopic contribution of R6G-d0 and R6G-d4 to the mixture spectra depends only on their concentration ratio, over a wide total concentration range.

To test whether a data analysis scheme can be derived for automatically data analysis, e.g. to determine the relative quantities of each of the isotopically different dyes, two spectral curve obtained with pure R6G-d0 and R6G-d4 are used as the bases set and on which all the curves obtained with mixtures are decomposed. For spectral decomposition with SERRS spectra were performed using only the 545 cm⁻¹ to 674 cm⁻¹ spectral region. The linear baseline of each truncated spectrum was automatically determined from a fit of the first and last three data points in the above spectral window, and the resulting baseline subtracted data matrix is represented as D. The truncated and baseline subtracted spectral matrix S containing the pure R6G-d0 and R6G-d4 spectra was obtained in the same way from the corresponding single component SERRS spectra. After intensity calibration (as described in Experiment Section), the relative concentration of R6G-d0 and R6G-d4 in the matrix C was readily determined using the following least square spectral decomposition where superscript t and −1 represents matrix transpose and inverse respectively.

C=D·S^{t}·(SS^{t})⁻¹ Eq 4

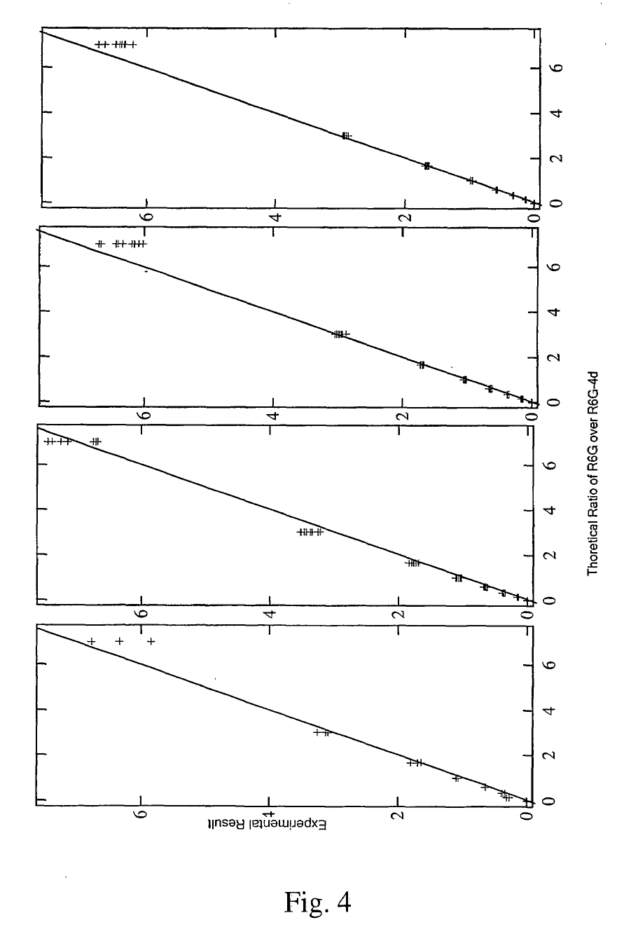

However, as described in the background, the SERRS intensity depends not only on concentration, but also on the characteristics of the colloidal solution. To properly test the IEIS method, the component spectra in matrix S were acquired with one batch of colloid solution, and the SERRS spectra used for prediction were obtained with different batches of colloidal solution. Furthermore, samples of total R6G concentrations of 200 nM and 200 pM were used to further test the robustness of the IEIR method. The results are shown in FIG. 4 in which the prediction of the ratio of R6G-d0/R6G-d4 based on the spectral signature of the mixture with the theoretical composition shown as values in the X coordinate. From left to right, the total concentrations of R6G-d0 and R6G-d4 are 1×10⁻¹⁰M, 1×10⁻⁹M, 1×10⁻⁸M, 1×10⁻⁷M respectively. FIG. 4 shows the predicted percentage of R6G-d0 with SERRS spectra obtained from mixtures with (a) the first batch and (b) the second batch of colloidal solutions (and the pure component spectra in matrix S were acquired with the third batch of colloidal solution with a R6G concentration of 200 nM). The average and standard deviations of each data point in both plots were obtained from 10 SERRS measurements, five with total R6G concentration of 200 nM and another five with a total R6G concentration of 200 pM. Similar results were obtained when the pure component spectra in matrix S were acquired with any batch of the Ag colloidal solution. Similar results were also obtained using SERS (rather than SERRS) measurements with a total R6G concentrations of 20 nM, 200 nM and 2 μM (and separate batches of colloidal solution used for calibration at a 20 nM R6G concentration and testing at the three different concentrations)

An average error of 2.1% in the concentration ratio obtained when the IEIS and the analyte were of the same concentration. When all the SERRS and SERS data were considered for samples in which the concentration difference between R6G-d0 and R6G-d4 is less than or equal to 3, the average concentration ratio prediction errors were 2.8%. Furthermore, the robustness of IEIS method is clearly demonstrated with the high reproducibility data shown in FIG. 4, obtained from different batches of colloidal solution of a 1000-fold analyte total concentration range. Moreover, by combining SERS and SERRS measurements, the concentration of R6G-d0 can be accurately quantified over a concentration range of four orders of magnitude from 200 pM to 2 μM.

However, when the concentration difference of analyte and its IEIS is greater than about a factor of 3, somewhat larger concentration ratio prediction errors may be obtained. Thus, to ensure an accurate quantification, it is preferable to pre-estimate the concentration of the analyte and to make sure similar amount of IEIS and analyte were mixed prior to the SERS/SERRS acquisition. Note that this can readily be done by pre-testing the analyte solution of interest using several IEIS solutions each differing by a factor of 10 in IEIS concentration.

As a further measure of the improvement in quantization obtained using the IEIS method, we attempted to correlate the absolute intensity of the SERRS signal of the analyte of R6G-d0 with its concentration (without using an IEIS reference). The normalized SERRS signal intensities at 615 cm⁻¹ obtained at each concentration with different batches of silver colloidal solution are shown at Table 2.

TABLE 2
Normalized SERRS peak intensity at 615 cm⁻¹ for R6G-d0
at different concentrations.

| | |
|---|---|
| Silver | Concentration of R6G-d0 |

| Colloidal | 200 pM | 2 nM | 20 nM | 200 nM |
|---|---|---|---|---|
| Batch One | 1.00 | 6.11 | 41.32 | 651.55 |
| Batch two | 2.16 | 4.70 | 35.76 | 1428.11 |
| Batch three | 1.27 | 5.83 | 19.86 | 540.06 |

In the normalization step, the difference in the integration time for different samples was compensated and the peak intensity for the SERRS spectrum obtained with the first batch of colloidal solution and with 200 pM of R6G-d0 was set to 1.00. Evidently, linearity of the SERRS data obtained with any one batch of the colloidal solution was quite poor as was the reproducibility of the data obtained with different batches. The relative prediction error obtained from such correlations was large as 300% (as determined by attempting to predict the concentration using SERRS data for a one batch of colloidal solution that was predicted using an intensity/concentration correlation derived from data acquired using another batch of colloidal solution)

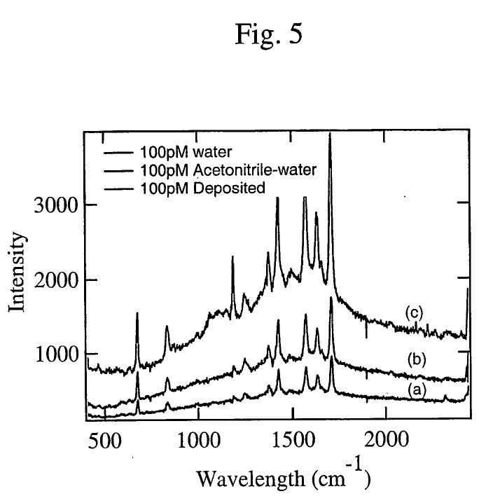

Since most of the HPLC or other separation methods may use mixtures of different solvents, to test how common solvents or SERRS detection schemes affect SERRS spectra, SERRS spectra of R6G-d0 dissolved in different ratio of acetonitrile/water mixture are obtained. Shown in FIG. 5 are SERRS spectra of R6G-d0 obtained at different solvent levels and without solvent (solvent evaporated). SERRS spectra of R6G. Spectrum (a), and (b) are obtained at R6G of 100 pM with solvent of water (a) and mixture of acetonitrile/water (25/75) (b) respectively. Spectra (c) are obtained by depositing 4 ul of the solution for spectra (b) onto quartz substrates. The acquisition time is 1 second.

The results shown in FIG. 5 demonstrate that the methods of the present invention have sufficient sensitivity to be directly coupled with a chromatographic separation process, either by detecting analytes in the separated fractions or directly on the chromatographic substrate. Further, variations in solvent and detection scheme for R6G-d0 and R6G-d4 do not affect the experimental results. The methods of the present invention, using isotopic substituted SERS or SERRS active labels including selected nucleic acid specific functional groups and Raman spectroscopy for comparative gene expression, can be used, for example, to identify variations in gene expression of biological system under various states such as genetics, aging, disease, drugs and/or environmental factors.

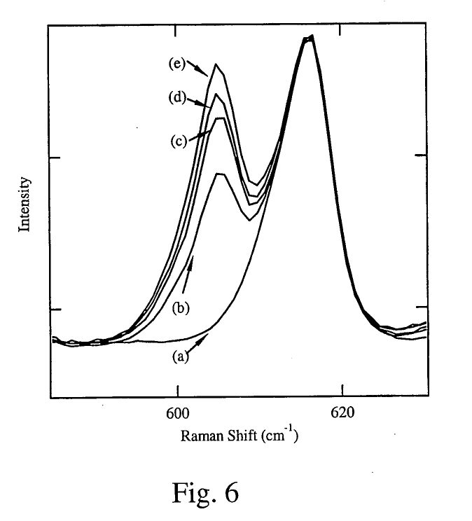

The in-situ addition of the IEIS into the analyte solution that has been pre-equilibrated with a colloidal solution can also be done, but this procedure proved to be less reliable than first pre-mixing the analyte and IEIS and then adding the colloidal solution. However, the results revealed interesting dynamics associated with the competition of the analyte and IEIS for SERS/SERRS active sites of the colloidal particles. More specifically, these tests were performed by first pre-mixing an analyte solution with a SERS active colloidal solution, to produce a final R6G-d0 concentration of 100 nM. After the 3 minutes of pre-equilibration, an equal amount of the IEIS (R6G-d4) was added into above mixture and a series of SERS spectra were acquired at different time after the IEIS was added. The resulting evolution of the Raman feature in the 585 cm⁻¹ to 630 cm⁻¹ spectral window is shown in FIG. 6, along with spectrum (e) obtained from sample in which equal amount of R6G-d0 and R6G-d4 were pre-mixed before adding the colloidal solution. Thus, these spectra reveal that when the IEIS is added to a pre-equilibrated analyte/colloid mixture significant time is required for the final mixture to equilibrate, and even after 4 hours the intensity of the IEIS has not yet reached equilibrium. Clearly such a procedure cannot readily be used to quantify the analyte concentration, but it could be used to study the kinetics associated with the analyte colloid binding and exchange reactions.

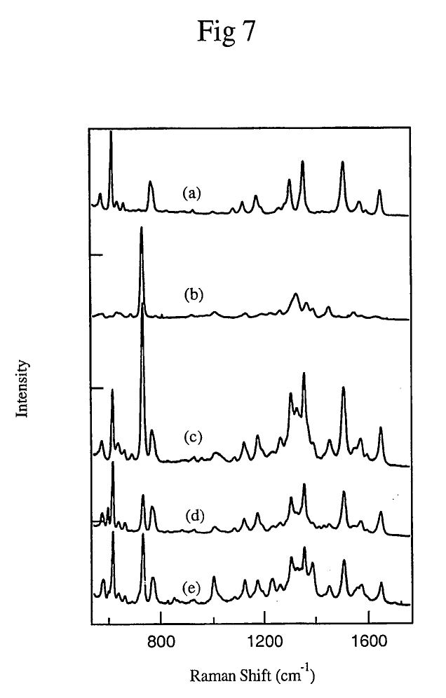

As yet another demonstration of the advantages of the IEIS method, we performed similar experiments using adenine rather than R6G-d4 as an internal SERS standard for R6G-d0 concentration measurements. More specifically, we pre-mixed a series of solutions each with a 1:100 ratio R6G-d0 to adenine (because adenine has about a 100 times weaker SERS signal). The R6G-d0 and adenine were pre-mixed before adding the colloidal solution. SERS measurements were performed with final R6G-d0 concentrations of 10 nM, 100 nM, and 1 uM. FIG. 7 shows the resulting SERS spectra obtained with (a) pure R6G-d0, (b) pure adenine, (c) 10 μM adenine and 100 nM R6G-d0, (d) 1 μM adenine and 10 nM R6G-d0, and (e) 100 nM adenine and 1 nM R6G-d0. In contrast to the results shown in FIG. 3A, where the relative SERS contribution of the analyte and its internal standard depends only on their relative concentration, the relative SERS contribution of R6G-d0 and adenine varied significantly at different concentrations (even though all the solutions had the same concentration ratio). Although the mechanism for the observed variations are not yet known, these results may indicate that using a different molecule as a SERS internal standard is not nearly as effective as using an IEIS derivative of the analyte of interest.

Although the IEIS method may find many different types of applications, it may prove particular valuable for detection gene expression patterns and for comparative proteomics studies, as in both these applications it is important to accurately quantify the relative concentrations of biomolecules derived from different sources. These applications may use tags and IEISs to label biomolecules derived from different samples, and then determine the relative concentration (amount) in each sample using the IEIS method. Key advantages of this approach over other tagging methods derive from the fact the chemical properties of the sample and reference are virtually identical, thus minimizing quantization errors associated with differential optical properties and tagging efficiencies, or differences in substrate binding and/or chromatographic retention.

While the forgoing studies generally employed R6G, other isotopically edited dyes may be used in the same processes. Xanthene dyes can be functionalized via formation of tertiary amides through the 2′-carboxylic group. Secondary amines have been prepared to achieve this with an aim of modifying the other terminal of the linker for obtaining molecular entities that can undergo coupling with lysines or cysteines of proteins or 5′-amino/thio modified nucleic acids. These isotopically edited dyes are readily obtainable fluorescent probes, which are useful for labeling biomolecules. The ISLRs of the present invention are not only valuable in detecting and quantifying populations of biomolecules using SERS and SERRS, they also are capable of being used in the field of fluorescence.

The linkers for attaching specific end groups for tagging with biomolecules can be built in during the synthesis of triarylmethane dyes. This can be achieved through a non-symmetric N,N-disubstituted aniline with one alkyl chain bearing a masked functional group for cysteine (SH) or lysine (NH₂) tagging. Synthesis of Benzotriazole azo dye with specific linkers has been demonstrated in the literature. With any of the dyes, the SERS or SERRS spectrum from a bioconjugate is expected to be substantially identical with those from the corresponding fluorophores, since the chromophores are separated from the linking group by an alkyl spacer. The resultant bioconjugates may be used to detect and quantify biomolecules present at picomolar concentrations using the SERS or SERRS measurement platform.

Based on the mini mal dissimilarity in chemical composition of the isotopic variants used in the present invention, two or more such isomers will have almost identical absorption characteristics, similar chromatographic properties, as well as almost identical orientation on the metal particles or surfaces employed in the SERS or SERRS interaction. As a result, the magnitude of surface enhancements obtained in SERS or SERRS will be nearly identical for the isotopic variants of an SERS or SERRS active dye. Both relative quantification and absolute quantification of any tagged analyte using the ISLRs of the present invention down to nanomolar and picomolar concentrations with high accuracy and repeatability.

The foregoing descriptions of specific embodiments of the present invention have been presented for purposes of illustration and description. They are not intended to be exhaustive or to limit the invention to the precise forms disclosed, and many modifications and variations are possible in light of the above teaching. The embodiments were chosen and described in order to best explain the principles of the invention and its practical application, to thereby enable others skilled in the art to best use the invention and various embodiments with various modifications as are suited to the particular use contemplated. It is intended that the scope of the invention be defined by the claims appended hereto and their equivalents.

What is claimed is:

1. A labeling reagent comprising an isotopically substituted reagent having a distinct SERS or SERRS spectral signature.

2. The labeling reagent of claim 1 wherein the reagent comprises a deuterated organic dye.

3. The labeling reagent of claim 1 or 2 wherein the organic dye is selected from xanthene dyes, triarylmethane dyes, and azo dyes.

4. The labeling reagent of claim 2 wherein the deuterated organic dye is obtained from a condensation reaction of a deuterated precursor.

5. The labeling reagent of claim 2 wherein the deuterated organic dye is obtained by isotopic exchange of aromatic protons between the organic dye and a deuterated acidic media.

6. A method quantitatively evaluating an analyte comprising the steps of:
labeling a portion of an analyte with an isotopically substituted SERS or SERRS active reagent,
subjecting the analyte to a separating regimen, and detecting the labeled portion with a Raman spectral detector.

7. The method of claim 6 wherein the labeling step comprises the steps of:
mixing a sample containing the analyte of interest with an isotopically edited internal standard of known concentration, and
incubating the mixture with a SERS active reagent to form a bioconjugate.

8. The method of claim 6 or 7 wherein the analyte of interest is a gene fragment.

9. The method of claim 6 or 7 wherein the analyte of interest is a protein or protein based biomarker.

10. The method of claim 7 wherein the detecting step is periodically repeated during the incubating step to quantify the reaction kinetics associated with the analyte colloid binding and exchange reactions.
